(19) United States
(12) Patent Application Publication
(10) Pub. No.: US 2026/0305925 A1
(43) Pub. Date: Oct. 8, 2026

(54) RETROGRADE TETHERED CAPSULE ENDOMICROSCOPY SYSTEMS AND METHODS

(71) Applicant: The General Hospital Corporation, Boston, MA (US)

(72) Inventors: Guillermo Tearney, Boston, MA (US); Mikayla Tinus, Beverly, MA (US); Evan Sevieri, Bedford, MA (US); WeiXuan Lai, Cambridge, MA (US); Du-Ri Song, Boston, MA (US); Fang Hou, Boston, MA (US); Constantinos Pitris, Boston, MA (US); Andrew Thrapp, Somerville, MA (US)

(21) Appl. No.: 19/100,916

(22) PCT Filed: Aug. 3, 2023
(86) PCT No.: PCT/US2023/071600

§ 371 Date: Feb. 3, 2025

Related U.S. Application Data
(60) Provisional application No. 63/394,940, filed on Aug. 3, 2022.

(51) Int. Cl. A61B 1/04; A61B 1/00; A61B 1/07; A61B 1/12; A61B 1/31; A61M 25/00

(52) U.S. Cl. CPC A61B 1/041; A61B 1/000094; A61B 1/000096; A61B 1/00082; A61B 1/00096; A61B 1/0011; A61B 1/00117; A61B 1/00119; A61B 1/0016; A61B 1/07; A61B 1/126; A61B 1/31; A61M 2025/006

(57) ABSTRACT

A system for performing retrograde tethered capsule endomicroscopy, including: a capsule including at least one outwardly-facing helical thread; a drive shaft coupled to the capsule and configured to rotate the capsule; and an optical system disposed within the capsule and configured to obtain circumferential imaging information. A method for performing retrograde tethered capsule endomicroscopy, comprising: providing a capsule comprising at least one outwardly-facing helical thread and an optical system disposed within the capsule; rotating the capsule using a drive shaft coupled to the capsule; and obtaining circumferential imaging information using the optical system disposed within the capsule.

### CROSS-REFERENCE TO RELATED APPLICATIONS

The present application is based on and claims priority from U.S. Patent Application Ser. No. 63/394,940, filed on Aug. 3, 2022, the entire disclosure of which is incorporated herein by reference.

### STATEMENT REGARDING FEDERALLY SPONSORED RESEARCH

N/A.

### BACKGROUND

Optical Coherence Tomography (OCT) is a noninvasive optical diagnostic tool that provides high-resolution microscopic images of tissues. In order for the light within an OCT device to adequately penetrate into tissue, the probe must be delivered inside of the human body. Although various designs of OCT devices have been made for probing the human body, further developments are needed to provide access for OCT imaging in various parts of the body.

### SUMMARY OF THE INVENTION

Accordingly, new apparatus, systems, and methods for performing retrograde tethered capsule endomicroscopy are desirable.

Various embodiments of the invention provide a Retrograde Tethered Capsule Endomicroscopy (R-TCE) catheter. The catheter may use optical coherence tomography (OCT) to image the lower gastrointestinal (GI) tract by insertion of a tethered capsule containing micro-optics into the anus. Implementations of the catheter may be used to diagnose diseases in the lower GI tract, such as Crohn's disease, and to screen for colorectal cancer including adenomas.

Embodiments of the R-TCE catheter include a rigid capsule body with a silicone external threaded layer connected to the distal end of a long, semi-rigid driveshaft. The driveshaft is surrounded by a smooth external sheath. The central channel of the driveshaft contains a flexible inner sheath which houses and protects insulated electrical wires and an optical fiber. The driveshaft functions to drive the capsule via a motor at the proximal end where it is attached to a separate drive system, the Retrograde Tethered Capsule Endomicroscopy Drive System (R-TCE-DS). The drive system rotates the driveshaft which translates the rotational movement and force to the threaded capsule. The threads transform the rotational movement to axial movement which progresses the capsule through the lower GI tract. To remove the device, the drive system switches to rotate in the opposite direction. The rigid capsule body on the distal end of the driveshaft contains micro-optics, including a ball lens connected to the optical fiber and a reflective prism, and a micro-motor to spin the prism. The electrical wires may be soldered to the motor and provide power to the motor from the termination on the catheter cap. The rotating prism allows the OCT light to reach the capsule wall around the entire circumference, which creates a 360° image that can be displayed on the imaging system.

The catheter cap on the proximal end of the driveshaft contains the optical and electrical terminations and the locking mechanism for connection to the drive system. The inner catheter cap is threaded onto the driveshaft, and the outer catheter cap is added later. The most proximal end of the inner catheter cap houses the optical and electrical terminations of the fiber and wires, and a bearing (e.g., a ball bearing) may be placed on the distal end of the inner cap to isolate rotation to only the inner cap and driveshaft/capsule once the outer cap is placed on. The fiber is epoxied into an SC/APC connector and the ferrule is polished for a smooth finish, and the electrical wires are soldered into spring loaded contact pins. The outer catheter cap is epoxied around the inner catheter cap, and a locking mechanism that connects to the drive system allows the inner cap to rotate and holds the outer cap in place.

In one embodiment, the invention provides a system for performing retrograde tethered capsule endomicroscopy, including: a capsule including at least one outwardly-facing helical thread; a drive shaft coupled to the capsule and configured to rotate the capsule; and an optical system disposed within the capsule and configured to obtain circumferential imaging information.

In another embodiment, the invention provides a method for performing retrograde tethered capsule endomicroscopy, including: providing a capsule including at least one outwardly-facing helical thread and an optical system disposed within the capsule; rotating the capsule using a drive shaft coupled to the capsule; and obtaining circumferential imaging information using the optical system disposed within the capsule.

In yet another embodiment, the invention provides a method for polyp detection, including: obtaining, using a processor, an image of a sample; determining, using the processor, a correlation of the derivative (COD) bandwidth for the image; determining, using the processor, a scattering coefficient for the image; determining, using the processor, an angular scattering coefficient for the image; and training, using the processor, a classifier based on the COD bandwidth, the scattering coefficient, and the angular scattering coefficient to predict a polyp type in the sample.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a schematic of the R-TCE and the OFDI console, where the inset shows the capsule without the external threads for clarity.

FIG. 1A shows a close-up image of the capsule indicating (from left to right) the diameters of the outer sheath (2.1971 mm), external silicone layer (13 mm), external imaging window (11 mm), and silicone threads (16 mm).

FIG. 2 shows a schematic of the R-TCE optical probe subsystem.

FIG. 3 shows a schematic of the distal scanning subsystem.

FIG. 4 shows a schematic of the internal capsule subsystem.

FIG. 5 shows a schematic of the external capsule subsystem.

FIG. 6 shows a schematic of the driveshaft subsystem.

FIG. 7 shows a schematic of the catheter cap.

FIGS. 8A-8C show aspects of a retrograde tethered capsule endomicroscopy device. FIG. 8A shows a schematic of the retrograde tethered capsule endomicroscopy device. FIG. 8B shows a tether cross-section. FIG. 8C shows a magnified view of the capsule. SMF: single mode fiber; dc: drive cable; s: tether sheath; dcca: drive cable-capsule attachment; t: silicone threads; bl: ball lens; mnt: motor mount; m: motor; w: imaging window; p: prism; O/E connector: optical/electrical connector.

FIG. 9 shows a magnified view of the capsule. SMF: single mode fiber; dc: drive cable; s: tether sheath; dcca: drive cable-capsule attachment; t: silicone threads; bl: ball lens; mnt: motor mount; m: motor; w: imaging window; p: prism; O/E connector: optical/electrical connector.

FIG. 10A shows a photograph of a prototype (top panel) and a diagram (bottom panel) of a distal tip based on a guidewire. FIG. 10B shows a photograph of a prototype (top panel) and a diagram (bottom panel) of a distal tip based on a tapered spring.

FIG. 11 shows a diagram of an R-TCE capsule being treated with an etching plasma to improve the antifouling performance of the surface of the capsule.

FIG. 12 shows an R-TCE OCT-Therapy system.

FIG. 13 shows an R-TCE OCT-Therapy system which includes one waveguide and a ball lens to deliver both types of electromagnetic radiation to the tissue.

FIG. 14 shows an R-TCE OCT-Therapy system which includes two waveguides and a focusing/collimating lens, where the lens focuses the OCT laser and collimates the therapy light.

FIG. 15 shows an R-TCE OCT-Therapy system which includes one waveguide and a focusing/collimating lens, where the lens focuses the OCT laser and collimates the therapy light.

FIG. 16 shows an R-TCE OCT-Therapy system which includes one waveguide and a tunable lens, where the tunable lens can be adjusted to focus either the OCT laser or the therapy laser, depending on what mode the system is operating in.

FIG. 17 shows histology images of laser ablated colon tissue (swine colon). The left image was stained with nitroblue tetrazolium chloride (NBTC) to show the inactive cells. The right image was stained with H&E that shows the tissue area eliminated by laser.

FIG. 18 shows an enface projection (left panel) of a sample with normal and tubular adenoma portions and (right panel) a graph of standard deviation of correlation of the derivative (COD) bandwidth, showing a clear separation between the normal and tubular adenoma tissues.

FIG. 19 shows colon polyp scattering coefficients as determined by A-line fitting displayed as an enface projection.

FIG. 20 shows a 50 pixel square extracted from a us map

FIG. 21 shows a square extracted from us map (left panel) as shown in FIG. 20 along with a low pass filter (circle, center panel) extracted from the logarithm of the Fourier transform and a ring (right panel) extracted from the same log of the Fourier transform.

FIG. 22 shows a diagram of an Angular Scattering Index generating using a 50×50 pixel scanning square and dynamic fitting of an ellipsoidal aperture and ring.

FIG. 23 shows a diagram of an R-TCE system in communication with a computer system.

### DETAILED DESCRIPTION

In accordance with some embodiments of the disclosed subject matter, mechanisms (which can include apparatus, systems, and methods) for performing retrograde tethered capsule endomicroscopy are provided.

One way to deliver light into a tissue of interest is via a tethered capsule. Tethered capsules typically contain micro-optics that direct the optical beam to the side to focus on the walls of the tissue surrounding the capsule. The micro-optics are rotated by a micro-motor disposed within a housing such that circumferential data can be collected from the tissue surrounding the capsule. Electrical power for the motor may be provided through the tether via small, well-insulated wires that may be enclosed within the tether. The catheter wires may terminate in an electrical connector that plugs into a compact imaging system (CIS). All external parts that are in direct contact with the patient are generally made of biocompatible materials. Using such micro-optics along with interferometric imaging (e.g., optical coherence tomography, OCT, or variants such as OFDI) allows the tether on a tethered capsule device to be relatively thin and flexible since the tether only needs to accommodate the optical fiber and a few thin wires.

While tethered capsules have been deployed in the upper GI tract (e.g., in the esophagus, stomach, and duodenum), for various reasons this technology has not been put to use in other tissues including the lower GI tract (e.g., the colon and rectum). Although a tethered capsule can be delivered to the upper GI tract relatively straightforwardly in a manner that takes advantage of gravity and involuntary muscle movements (e.g., by having the patient swallow the capsule), delivering a tethered capsule all the way to the lower GI tract via the subject's mouth is not practical. The entire GI tract is about 18 feet (5.5 meters) in length and thus it would take a relatively long time for the capsule at the end of a tether to reach the lower GI tract. Furthermore, it may be difficult for the tether containing an optical fiber to withstand the many bends and curves along the entire length of the GI tract and the capsule could not be readily retrieved by pulling it backwards through the entire GI tract.

Although it is possible to access the lower GI tract via the anus, current procedures such as those used during colonoscopies are more involved than for tethered capsules and as such require sedation, making these procedures more complicated and costlier than those performed with tethered capsules. The complexity of accessing the lower GI tract via the anus is due in part to the need for the probe to be actively moved through the tissue, since a capsule attached to a thin, flexible tether will not move passively or via involuntary movements as is the case for swallowing. While a tethered capsule includes a relatively small capsule attached to the end of a thin and flexible tether, current probes for accessing the lower GI tract such as conventional endoscopes are relatively thick so that they can be guided into tissue and steered, which requires the patient to be sedated due to the discomfort that this may cause.

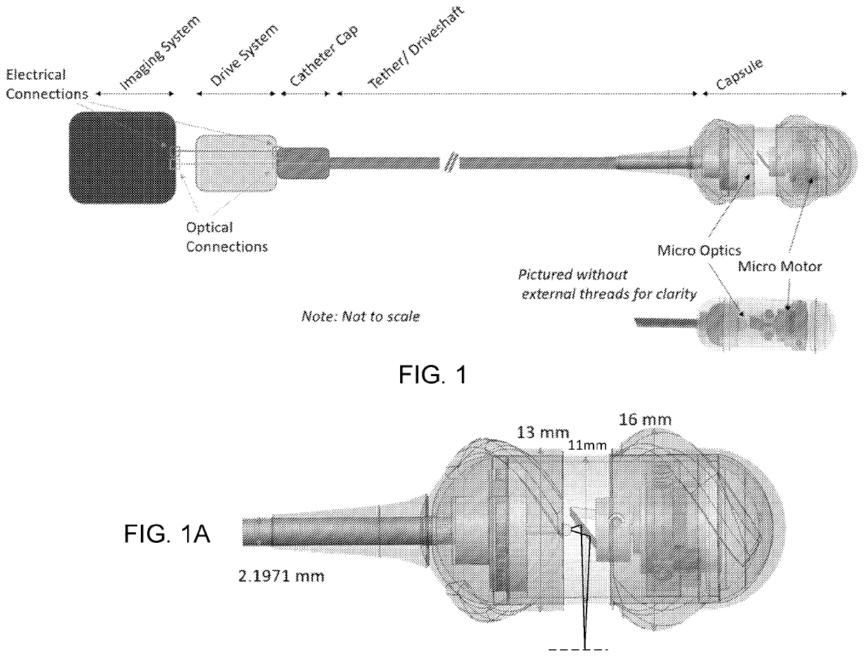

Thus, various embodiments disclosed herein provide a Retrograde Tethered Capsule Endomicroscope (R-TCE) catheter (FIGS. 1, 1A), which is a tethered capsule that is configured to move through tissue in an active manner and as such can be used to access the lower GI tract via the anus. The motive force for movement of the R-TCE capsule is provided by one or more outward-facing helical thread(s) on the capsule in combination with a rotational drive system coupled to the capsule by a semi-rigid driveshaft. In one embodiment, the semi-rigid driveshaft includes two coiled layers (which may be made of 304 stainless steel) in which one layer is wound in a right-handed direction and the other layer is wound in a left-handed direction. When the drive system rotates the capsule in a first rotational direction the capsule moves forward and advances into the tissue (e.g., into the anus and lower GI tract) due to engagement of the helical thread(s) of the capsule with the tissue. When the drive system rotational direction is reversed, the capsule moves in a reverse direction and backs out of the tissue, again due to engagement of the helical thread(s) of the capsule with the tissue. This arrangement allows the capsule to be driven and advanced in either direction with minimal discomfort using a relatively thin and flexible tether, which permits procedures to be conducted using the R-TCE on unsedated subjects.

In various embodiments, the R-TCE catheter includes a rigid capsule body with a resilient outer portion (e.g., made of silicone) containing an external threaded layer, where the capsule is connected to the distal end of a long, semi-rigid driveshaft. The driveshaft may be surrounded by a smooth external sheath. The central channel of the driveshaft may contain a flexible string-like inner sheath which houses and protects components such as the insulted electrical wires and optical fiber. In certain embodiments, the capsule is sized and shaped for insertion into the anus. The driveshaft functions to drive the capsule via a motor at the proximal end (i.e., the end closer to the operator and further from the subject) where it is attached to a separate drive system, the Retrograde Tethered Capsule Endomicroscope Drive System (R-TCE-DS) (FIG. 1). The R-TCE-DS rotates the driveshaft, which translates the rotational movement and force to the threaded capsule. The threads, when contacting the tissue of the GI tract, transform the rotational movement to axial movement (e.g., forward or reverse movement along the long axis) which progresses the capsule through the lower GI tract. To remove the device, the drive system switches to rotate in the opposite direction. The catheter materials that come into contact with the patient are biomedical or food grade. Although the threads shown in the embodiments of FIGS. 1 and 1A have a right-handed orientation, in other embodiments the threads may be left-handed.

In some embodiments, the drive system (R-TCE-DS) may be connected to a compact imaging system (e.g., an OCT-based system, FIG. 1) and thus endomicroscopic imaging using the R-TCE may be executed similar to upper GI tract OCT capsule endoscopy. In use, the capsule may be administered to the subject and, once inserted, the capsule travels in the lower GI tract through the threaded screw action driven by the R-TCE-DS.

While the capsule progresses through the colon, electromagnetic radiation (e.g., near infrared light) from the imaging system may be scanned on the tissue (e.g., the colon wall) surrounding the capsule by the rotation of the micro-motor's shaft enclosed inside of the capsule (note that rotation of the micro-motor shaft is separate from the rotation of the driveshaft subsystem). Light collected back from the tissue carries information about the internal structure of the tissue which may then be recorded, processed, and displayed in real time by the imaging system. The capsule progression and imaging may be operated manually by the capsule operator through the R-TCE-DS and the imaging system.

In certain embodiments, the Retrograde Tethered Capsule Endomicroscope (R-TCE) catheter may include a ball lens (BL) based optic system and a distal scanning (DS) mechanism, where the R-TCE may be “self-propelled” through the lower intestine (see FIGS. 1, 1A). In various embodiments, the R-TCE may be said to be “self-propelled” insofar as its rotation in a particular rotational direction, which is generated by the drive system, causes the capsule to advance into or out of the subject's tissue without requiring the tether to drive the capsule forward. Whether the capsule advances into or out of the tissue depends on the direction of rotation as well as the handedness of the helical thread(s) on the outside of the capsule.

### Imaging Console (OCT)

In various embodiments, R-TCE catheters may be designed to be used with existing imaging systems (CISs) such as the MGH OCT Imaging Console. The MGH OCT Imaging Console can either be electrically isolated from the catheter by using a battery-powered motor power unit to drive the micro-motor distal scanning optics or it can serve as a direct supply power to the motor power unit. This console also produces a lower optical power with a maximum of 35 mW at the same wavelength of 1310±50 nm on the tissue. This results in an estimated tissue radiant exposure of <0.01 mJ/cm², and this console has been approved for use in other studies. In various embodiments, the electromagnetic radiation delivered by the imaging system to the sample may include light having a wavelength in the UV, visible, and/or infrared region of the spectrum. The electromagnetic radiation may be delivered from the CIS/imaging system via an optical waveguide which may include an optical fiber such as a single mode fiber (SMF). In various embodiments, the imaging system may have a relatively short spectral bandwidth (~60 nm or less) compared to previous OCT imaging consoles used for esophageal imaging, which typically have provided an imaging depth of about 6 mm. The shorter spectral bandwidth was selected to enable 3D-OCT imaging of larger luminal structures, such as the colon, and to provide a longer-range depth imaging (12 mm imaging depth).

### OCT Retrograde Tethered Capsule Endomicroscope (R-TCE)

In certain embodiments, the catheter may be designed for intraluminal navigation and imaging of the human lower gastrointestinal tract. The R-TCE catheter includes a rigid capsule (e.g., made of PMMA) with an external threaded layer (e.g., made of silicone) connected to the distal end of a long, semi-rigid driveshaft, where the driveshaft may be surrounded by a smooth outer sheath. The central channel of the driveshaft contains a flexible string-like inner sheath which houses and protects insulated electrical wires and an optical fiber. The size and shape of the capsule and tether (FIG. 1A) allows for its insertion into the anus. The driveshaft allows the capsule to be driven through a motor at the proximal end. The R-TCE device connects to a drive system (R-TCE-DS) with the catheter cap which houses optical and electrical terminations. The drive system rotates the driveshaft which translates the rotational movement and force to the threaded capsule, allowing for the translation of rotational to axial motion for the navigation of the capsule in the colon; the drive system is then switched in reverse for removal of the device. The catheter materials that come into contact with the patient are generally biomedical grade and/or food grade and the device has been tested for low pH resistance, tensile strength, and leaks and follows the relevant FDA guidance “Medical Devices; Gastroenterology-Urology Devices; Classification of the Colon Capsule Imaging System” and “Medical Devices; Gastroenterology-Urology Devices; Endoscope and accessories.”

As indicated above, in various embodiments the catheter is connected to a drive system, the Retrograde TCE Drive System (R-TCE-DS), which is connected to an IRB-approved imaging console which facilitates performing endomicroscopic imaging in a manner similar to the imaging procedures in place for previously approved protocols. In use, the capsule is administered to the subject and, once inserted, the capsule travels through the lower GI tract propelled by the threaded screw action driven by the R-TCE-DS. The driveshaft progression is controlled manually by the capsule operator through the R-TCE-DS and the imaging system.

### R-TCE Optical Probe Subsystem (OPS)

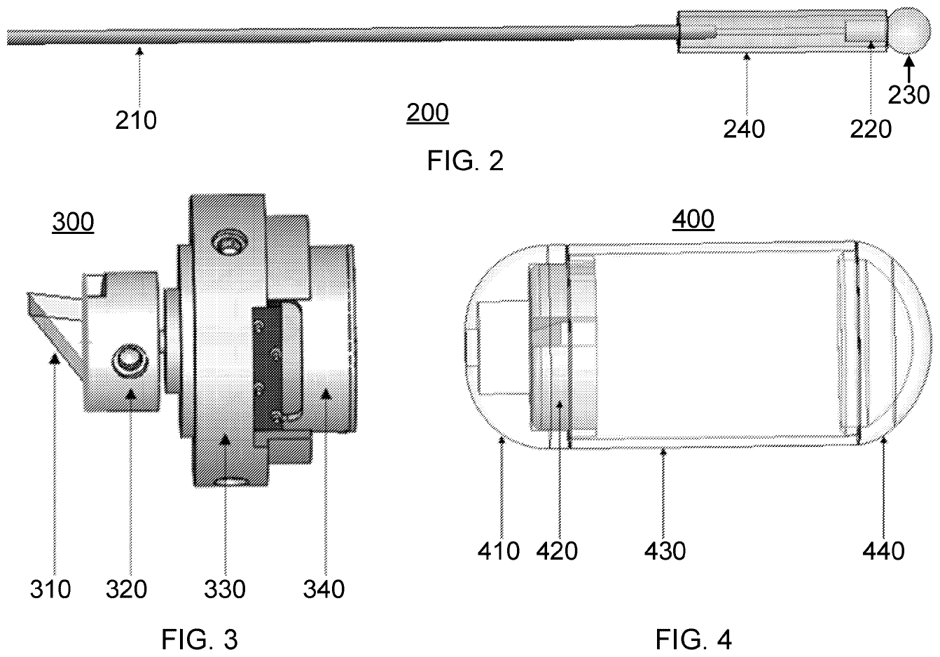

In certain embodiments, the optical components of the R-TCE Optical Probe Subsystem (OPS) 200 may include an optical single mode fiber 210 that is terminated with a glass spacer 220 and/or a ball lens 230 (FIG. 2). The optics are secured within the optical probe housing 240 or “hypotube” while the remaining length of fiber is enclosed within the Driveshaft Subsystem. On the proximal end of the OPS 200, an SC/APC optical connector is epoxied and polished after the fiber is threaded through the Driveshaft Subsystem. The SC is secured in the inner cap with epoxy and assembled in the Catheter Cap. The electromagnetic radiation (e.g., near infrared light) from the optical fiber may be focused by the ball lens to a point slightly beyond the outer surface of the side of the capsule after it is reflected by the prism, which is mounted on the angled shaft weight of the micro-motor. The beam coming out from the OPS 200 (e.g., from the ball lens) shines on the angled reflector which is mounted on the micro-motor sitting at the most distal end within the Distal Scanning Subsystem (FIG. 1A).

### R-TCE Distal Scanning Subsystem (Dist-SS)

In various embodiments, the components of the R-TCE Dist-SS 300 may include a prism 310, prism mount 320, micro-motor 340 as well as the motor centering piece 330, and/or wires (FIG. 3). The R-TCE Dist-SS 300 functions with the prism reflecting the light at a 90-degree angle. The prism 310 is mounted onto a shaft of the micro-motor 340 by the prism mount 320. In certain embodiments, the micro-motor 340 rotates at a rate of between 20 Hz-40 Hz, spinning the prism 310 and thusly allowing the light to scan in a circle. The micro-motor 340 is powered and controlled through the four electrically insulated wires that are distally terminated at the side of the micro-motor 340 and protected by a layer of epoxy. Each wire is threaded through the driveshaft subsystem and soldered to a spring-loaded contact pin on the proximal side which together form the electrical connector within the Catheter Cap.

### R-TCE Internal Capsule Subsystem

In certain embodiments, the Internal Capsule Subsystem 400 may include a proximal base 410, imaging window 430, distal cap 440, and/or base weight 420 (FIG. 4). The diameter and length of the capsule (see FIG. 1A) are comparable to those of FDA-approved, commercially available capsule endoscopes (Givens G2, OMOM Capsule Endoscope, Smart Pill) and colonoscopes (Olympus), which are widely used in clinical practice for probing the GI tract. The internal rigid capsule, for the purpose of assembling the catheter, includes three parts which are all made of food grade material: the proximal base 410, imaging window 430, and distal cap 440. These components make up the sealed, rigid capsule which contains the optomechanical components for OCT imaging. The proximal base 410 holds the base weight 420 which secures and centers the OPS 200. This connects to the imaging window 430 which surrounds the optics and has high optical clarity. The contour of the outer surface of the capsule ensures easy passage through lower GI tract. The distal portion of the imaging window 430 houses the Distal Scanning Subsystem 300 which is centered at the distal end to ensure proper light propagation. The distal cap 440 seals the capsule end with biomedical grade epoxy.

### R-TCE External Capsule Subsystem (ECS)

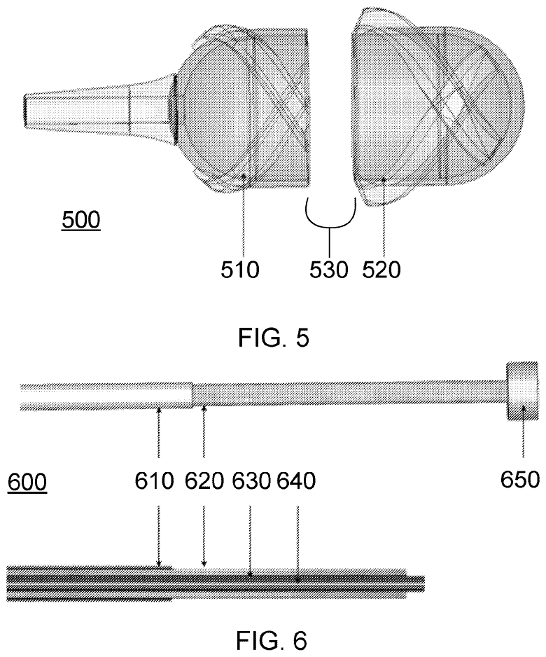

In some embodiments, the R-TCE External Capsule Subsystem 500 surrounds the rigid internal capsule (FIG. 5). This subsystem 500 may include one or both external threads 510, 520 which may be made of silicone and which function to transform the rotational force from the driveshaft and drive system to axial force and translation of the capsule through the colon. In certain embodiments, the threads may attach as proximal 510 and distal 520 layers allowing for a gap 530 therebetween to permit imaging. The proximal thread layer 510 has a tapered shape which functions as a strain relief, with its diameter gradually transitioning from a larger diameter which encompasses the outer surface of the capsule to a smaller diameter which encompasses the outer surface of the driveshaft (see FIG. 1A for exemplary diameter values). In particular embodiments, the R-TCE External Capsule Subsystem 500 may also include a hydrophilic coating which increases lubricity of the device to minimize tissue damage and patient discomfort.

### R-TCE Driveshaft Subsystem

In various embodiments, the R-TCE Driveshaft Subsystem 600 may include: a tether collar 650, which connects the driveshaft and inner sheath to the proximal base; an inner sheath 630, which houses the four wires and fiber; a bi-directional driveshaft 620; and/or an outer sheath 610, which isolates the rotation of the device from surface contact with tissue (FIG. 6). The tether collar 650, which may be made of brass, may be secured inside the capsule for improved interface strength.

### R-TCE Catheter Cap (CC)

The R-TCE catheter cap 700 on the proximal end of the driveshaft contains the optical and electrical terminations and the locking mechanism for connection to the drive system. The inner catheter cap is threaded onto the driveshaft, and the outer catheter cap is added later. The most proximal end of the inner catheter cap houses the optical and electrical terminations of the fiber and wires, and a ball bearing is placed on the distal end of the inner cap to isolate rotation to only the inner cap and driveshaft/capsule once the outer cap is placed on. The fiber is connectorized with an SC/APC connector. The outer catheter cap is epoxied around the inner catheter cap, and a locking mechanism that connects to the drive system can be added to allow the inner cap to rotate and holds the outer cap in place.

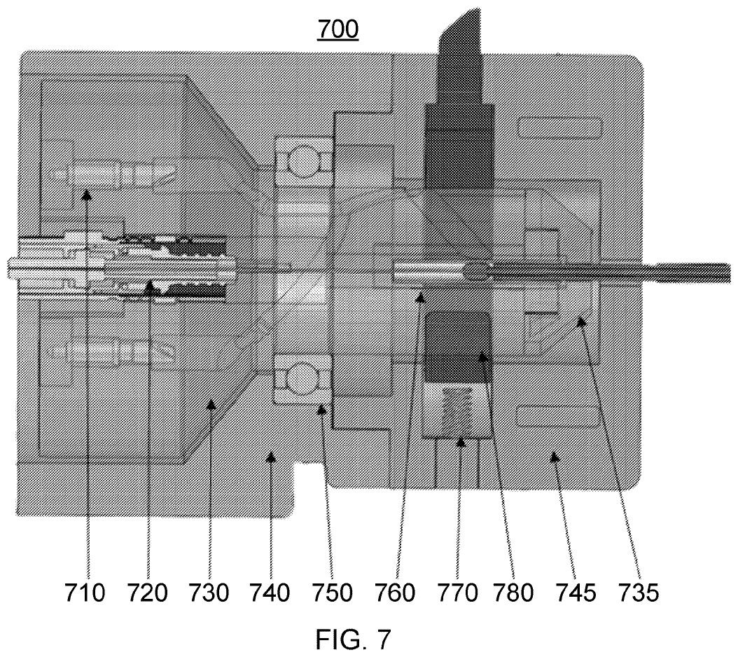

The R-TCE catheter cap 700 may serve as the mechanical, optical, and/or electrical connection to the Drive System (R-TCE-DS). In some embodiments, the R-TCE catheter cap 700 may include one or more electrical pins 710; an SC/APC fiber connector 720 which centers and terminates the OPS 200; an inner cap 730, 735; an outer cap 740, 745; a bearing 750; a hypotube 760; two springs 770; and/or a ring lock 780 (FIG. 7).

### Inner Cap

In some embodiments, the inner cap 730, 735 may house and protect the optical and electrical connections. The driveshaft subsystem's proximal end may be epoxied inside the inner cap 730, 735, where a channel separates the electrical wires while maintaining the central optics. The electrical wires may be secured into separate housings which may be soldered onto four separate electrical pins. The optical fiber and wires may be secured with a hypotube 760 which may have a notch to separate out the wires. The hypotube 760 may be epoxied to the driveshaft and the SC connector 720 where the fiber is terminated and angle polished. Both the electrical pins 710 and optical connector 720 are epoxied into a proximal face of the inner cap 730, 735. The inner cap 730, 735 is rigidly connected to the Driveshaft Subsystem and therefore rotates with the device.

### Outer Cap

In various embodiments, the outer cap 740, 745 may surround the inner cap 730, 735 and isolate the rotational motion for ease of use. The inner cap 730, 735 may rotate within the outer cap 740, 745 and may be stabilized and centered by the bearing 750. The ring lock 780, loaded by two springs 770, functions as the locking mechanism, and extends into a release button at the top of the outer cap 740, 745. In addition to the ring lock 780, the grooved external surface of the outer cap 740, 745 facilitates locking and proper alignment of the catheter cap within the Drive System.

In use, the R-TCE catheter uses optical coherence tomography (OCT) to image the lower gastrointestinal (GI) tract by insertion of a tethered capsule containing micro-optics into the anus. Various embodiments of the R-TCE device may be used to diagnose diseases in the lower GI tract (e.g., Crohn's disease and/or colorectal cancer), quantify inflammation, screen for adenomas, detect cancer in the lower GI tract, and/or to conduct colorectal cancer screening.

As disclosed herein, in certain embodiments the rigid capsule body on the distal end of the driveshaft contains micro-optics, which can include various focusing elements such as a ball lens or prism connected to the optical fiber and an angled reflector (e.g., a mirror or reflective prism) coupled to a micro-motor, to rotate the prism. In some embodiments, an ellipsoidal reflector may be used to guide light from the optical fiber to the tissue.

In various embodiments, an antireflection coating may be applied to the ball lens to reduce multiple imaging artifacts from the capsule wall. In some embodiments, the electrical wires may be soldered to the motor and provide power to the motor from the termination on the catheter cap. During use, rotating the angled reflector allows the electromagnetic radiation (e.g., OCT light) from the optical fiber to reach the entire circumference of tissue surrounding the capsule, which creates a 360° image that can be displayed on the imaging system (CIS).

In general, the threads (e.g., silicone threads) may not be fully optically transparent, and thus in certain embodiments providing an imaging window gap allows depth imaging with low light loss. In certain embodiments, the one or more threads of the capsule may include a gap to provide a circumferential imaging window that is aligned with the imaging optics to allow image data to be obtained from the tissue in an unobstructed manner. In other embodiments, the threads may extend the entire length of the capsule and other approaches may be used to compensate for possible distortion effects from the threads such as post-processing of image data or adjustment of the index of refraction of the thread material to more closely match that of the tissue.

In various embodiments, the R-TCE capsule may be made from a rigid material surrounded by a resilient outer layer that includes the one or more helical threads. While silicone is a suitable material for making the threads due to its biocompatibility and resilience, other resilient and biocompatible materials may be used instead of or in addition to silicone to form the outer layer of the R-TCE capsule. In particular embodiments, the threads may be integrally formed as part of the capsule body, which may be formed either from a rigid material or from a resilient or semi-resilient material. In some embodiments, an imaging window may be formed in the resilient outer layer by providing a central thinner portion that does not include the one or more threads (i.e., a gap in the thread(s)) or by providing the resilient outer layer as two separate pieces including proximal and distal layers (see FIG. 5). In particular embodiments, the one or more threads may helically encircle the capsule and may project out from the surface of the capsule and/or resilient outer layer by 1-5 mm and in one embodiment project out by 3 mm (FIG. 1A). The R-TCE capsule may include one, two, three, or other numbers of outwardly-projecting threads to facilitate movement of the capsule when it is rotated. The threads may be shaped and angled to varying degrees to best match the type of tissue to be traversed and to advance at a suitable axial rate for each rotation of the capsule. In general, the one or more threads may be or include a raised portion that projects outwardly from the capsule sufficiently far that the threads engage with the tissue to propel the capsule when it is rotated.

In certain embodiments, the rigid portion of the R-TCE capsule may have an outer diameter of between 8-15 mm, and in one particular embodiment has an outer diameter of 11 mm (FIG. 1A). In some embodiments, the outer layer may have a thickness in the non-thread regions of 0.5-3 mm, and in one embodiment has a thickness of 1 mm. In various embodiments, the portions of the outer layer which include threads may have a thickness of 1-5 mm and in one particular embodiment have a thickness of 3 mm. Finally, the outer sheath may be 2-3 mm in diameter, with one particular embodiment being 2.1971 mm. See FIG. 1A for exemplary diameters of various components of an embodiment of the R-TCE capsule.

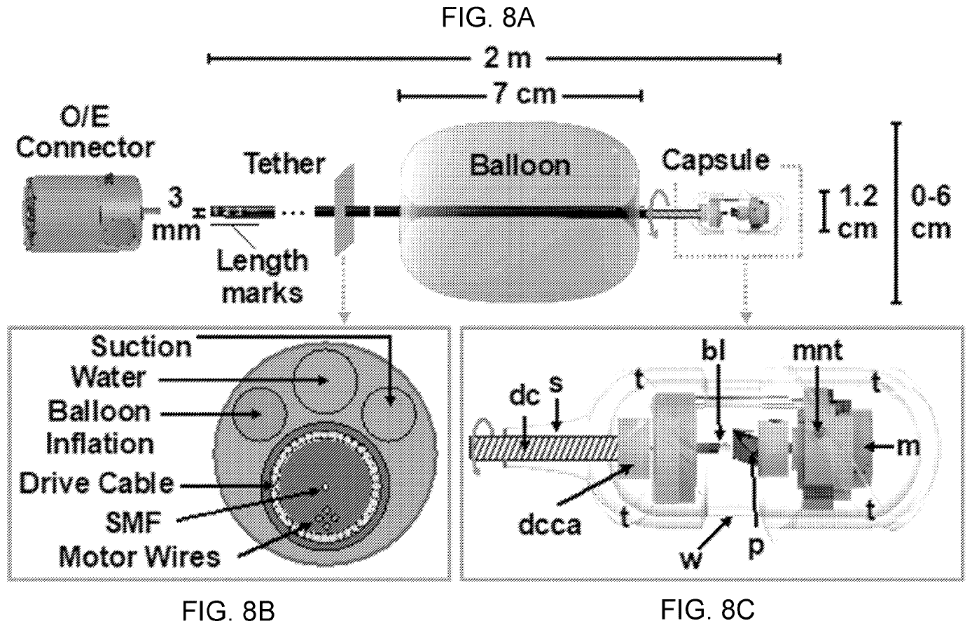

In various embodiments, a balloon may be attached close to the strain relief such that the balloon is adjacent to the capsule, for example below or on the bottom of the silicone cap (FIG. 8A). The balloon may be configured to expand within the colon to provide improved imaging quality. In particular, the balloon can be inflated (e.g., with gas such as air or liquid such as saline) during operation (e.g., during pullback imaging) of the capsule to center the capsule in the colon to obtain full image data from a full circumferential view of the surrounding tissue in a single pullback. In further embodiments, an infusion tube may be attached next to the driveshaft to deliver water/saline to the imaging window to remove debris.

FIG. 8B shows a cross-sectional view of the tether at the location indicated in FIG. 8A which is between the balloon and the proximal end. The cross-sectional view shows the drive cable with a single mode fiber (SMF) and motor power wires disposed therein; a balloon inflation channel (for delivery of a gas or liquid inflation fluid); a water delivery channel (for cleaning debris from the imaging window); and a suction channel.

FIG. 8C shows a close-up view of the capsule as indicated in FIG. 8A, where: dc: drive cable; s: strain relief; dcca: drive cable-capsule attachment; t: silicone threads; bl: ball lens; mnt: motor mount; m: motor; w: imaging window; p: prism.

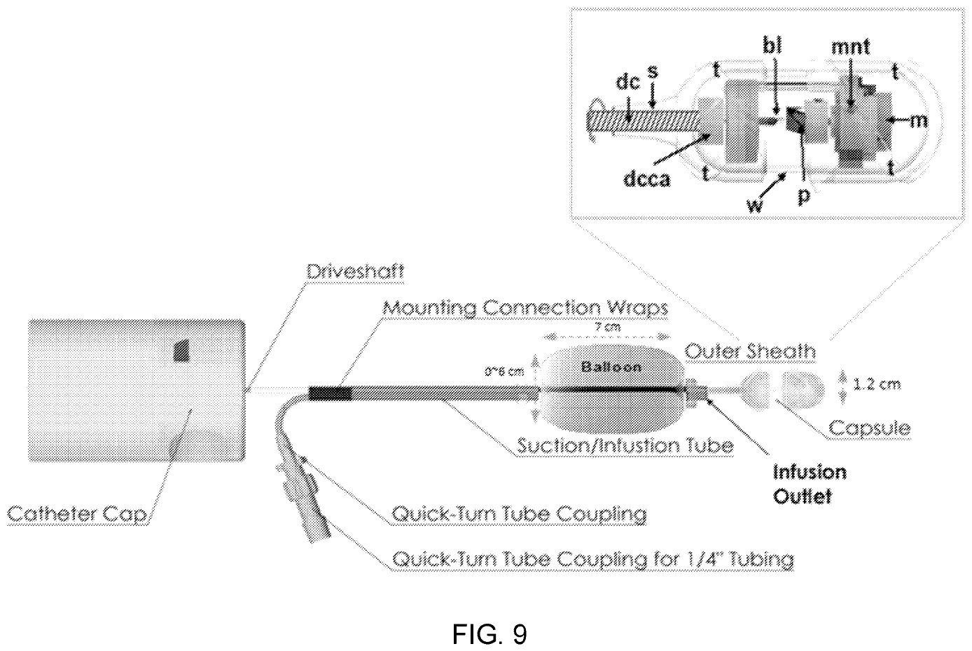

FIG. 9 shows a diagram of an R-TCE catheter with a balloon, where the diagram provides approximate sizes or size ranges for the axial length of the balloon (7 cm), diameter of the balloon (0-6 cm), and diameter of the capsule (1.2 cm). The diagram of FIG. 9 also shows the relative positions of the catheter cap, a suction/infusion tube with couplings and connection wraps, the driveshaft, the balloon, an infusion outlet, and the capsule (depictions of the various components are not to scale). Finally, the inset of FIG. 9 shows details of the capsule design.

In various embodiments, the balloon may expand to a diameter that is less than, equal to, or greater than an outer diameter of the R-TCE catheter. In the embodiment shown in FIG. 8A, the balloon is approximately 7 cm in the axial direction and has an outer diameter that is greater than the outer diameter of the R-TCE capsule. In various embodiments, the outer diameter of the inflated balloon is matched to the focusing distance of the R-TCE capsule optics (see FIG. 1A) so that the balloon spreads the tissue uniformly outward to a distance that is aligned with the focusing distance.

The use of external threads (e.g., silicone threads) on the outside of the capsule to transform the rotational movement to axial movement and to thereby facilitate OCT imaging inside of the lower GI tract has not previously been done. Further attaching a balloon to the capsule facilitates visualization of the relatively large diameter colon wall compared to the capsule size. Additionally, the R-TCE includes a rotating driveshaft that houses the inner sheath, or tether, similar to that used in upper GI tract capsules.

### Capsule Tip Attachments

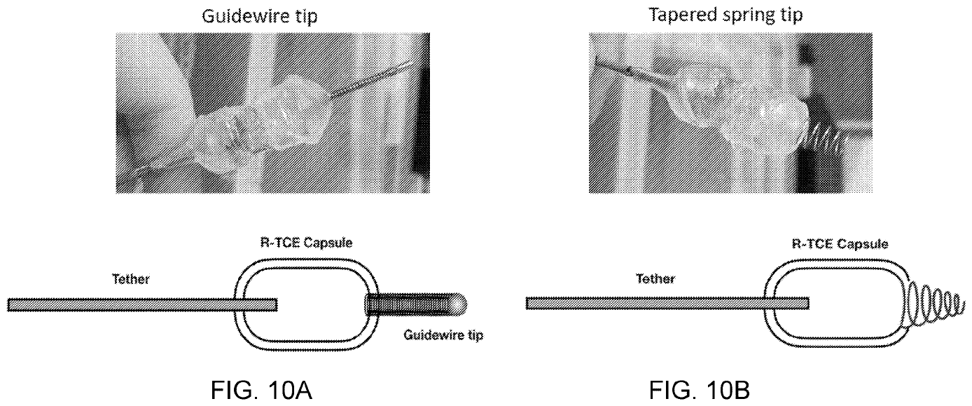

In some embodiments, the R-TCE capsule may include an extension at the distal end which helps keep the capsule from getting stuck at tight bends in the tissue (e.g., colon). In various embodiments, the extension may be a 25-50 mm distal tip of wire such as a guidewire (FIGS. 10A, 10B). In use, when the distal tip contacts the mucosal wall it causes the capsule to deflect, changing its angle and allowing it to traverse and continue to follow the course of the lumen. The distal tip can be a straight commercial GI guidewire, as shown in FIG. 10A, or a tapered spring shape, as shown in FIG. 10B. Here, the mechanical property of the distal tip can be selected by the guidewire tip's length, thickness, flexibility, or properties of the spring such as the shape, dimension (top and bottom), number of coils, and flexibility. As shown in FIG. 10, the distal tip may be relatively straight (FIG. 10A) or a tapered/conical shape (FIG. 10B).

The multi-use catheter generally undergoes a high-level disinfection (HLD) before the first use and after each use according to standard sterilization protocols (e.g., the sterilization methods in place at the Mass General Hospital (MGH) GI unit). In one embodiment, this protocol may be the same as that of MGH GI unit endoscopes and esophageal manometry study catheters (ESMO), both of which are passed through the upper GI tract and removed.

### Plasma Treatment

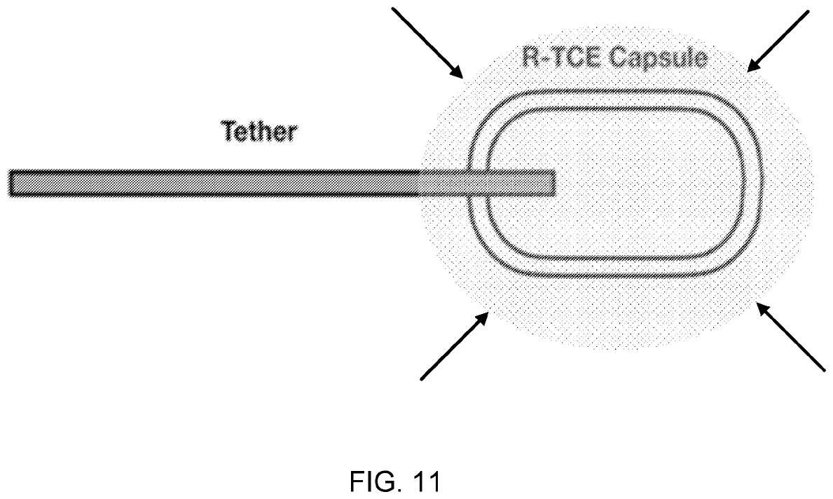

In some embodiments, the R-TCE capsule may be pretreated to reduce fouling of the capsule during use to make subsequent cleaning and sterilization easier. In various embodiments, a rigid capsule made of polymethyl methacrylate (PMMA) can be treated with a O₂/CF₄ plasma (see FIG. 11, indicated by arrows) to modify the R-TCE capsule wall by etching. As a result of the plasma treatment, the O₂ gas increases the hydrophilicity of the surface and the CF₄ case increase the hydrophobicity of the surface, which together improves the antifouling performance. Good antifouling properties and hydrophilicity can both be achieved in the plasma-treated PMMA capsule with CF₄ content ranging from 20% to 40%.

### Ablation

In various embodiments, the R-TCE device may be used to treat pre-cancer lesions, including colon polyps, in the colon with OCT-guided laser ablation. In such embodiments, the device includes an OCT imaging system and a therapy laser integrated into the imaging system. In certain embodiments, the OCT system may include an OCT imaging console and an OCT-therapy capsule, where the capsule may include a waveguide (such as a single-mode fiber or a multimode fiber), or multiple waveguides (such as a multi-core fiber), that delivers the therapy light into the OCT capsule. The capsule may also include optical components that further deliver the light to the side of the capsule to the identified lesion, such as those components disclosed herein, where the optical components for therapy may be shared with the OCT imaging components.

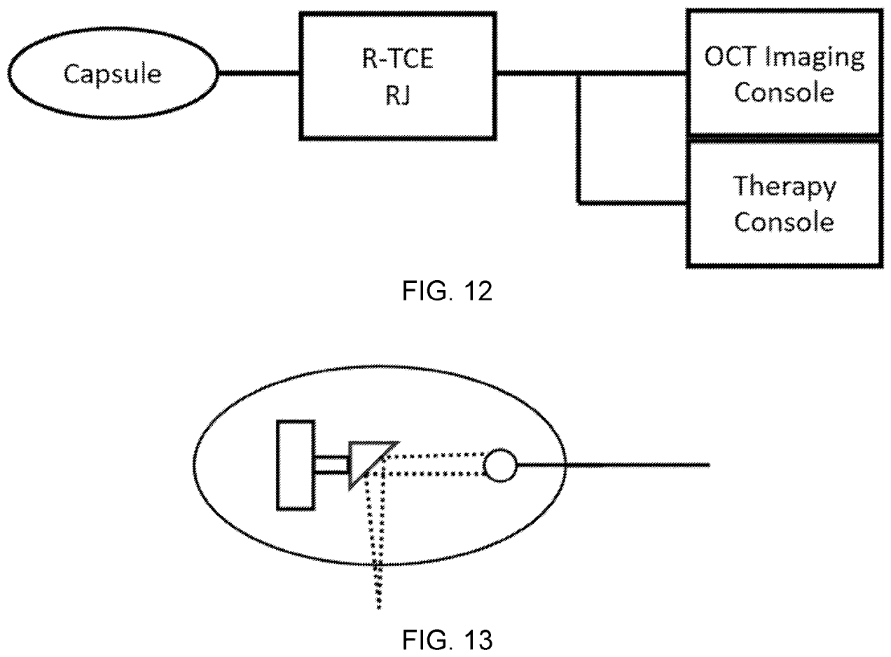

FIG. 12 shows an R-TCE OCT-Therapy system, which may be based on an R-TCE such as those disclosed above which has an additional therapy laser in the imaging console or in a separate therapy console. Thus, in certain embodiments the R-TCE OCT-Therapy system may include a first electromagnetic radiation source as part of an imaging console (e.g., the OCT imaging console in FIG. 12) and a second electromagnetic radiation source as part of a therapy/ablation console (e.g., the therapy console in FIG. 12). One embodiment of the therapy capsule includes a waveguide, a ball lens, and a rotational reflector to deliver the light, where the ball lens focuses both the OCT light and the therapy light (both of which are indicated by dotted lines) on the tissue (FIG. 13).

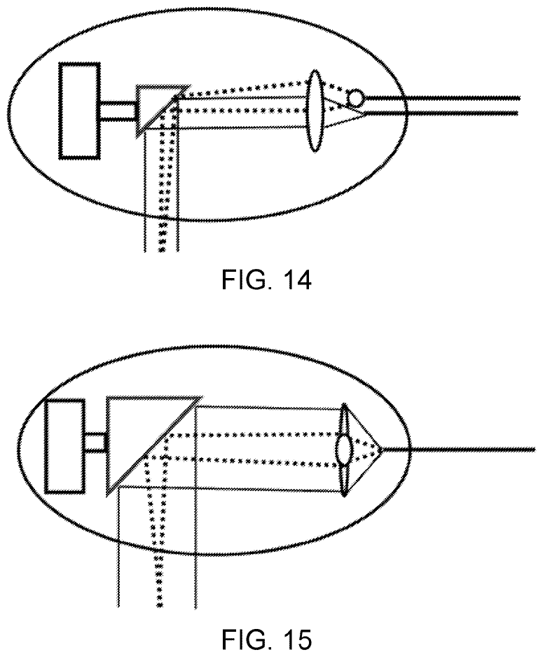

Another embodiment of the therapy capsule includes a second waveguide (in addition to the OCT waveguide) to deliver electromagnetic radiation for therapy, a focusing/collimating optical module, and a rotational reflector to deliver the light (FIG. 14). The focusing/collimating optical module focuses the OCT light (dotted lines) for imaging and collimates the therapy light (solid lines) to cover a large area of the tissue for ablation. Still another embodiment of the therapy capsule (FIG. 15) includes a single waveguide which delivers OCT and/or therapy light to a focusing/collimating lens, where the lens focuses the OCT laser (dotted lines) and collimates the therapy light (solid lines).

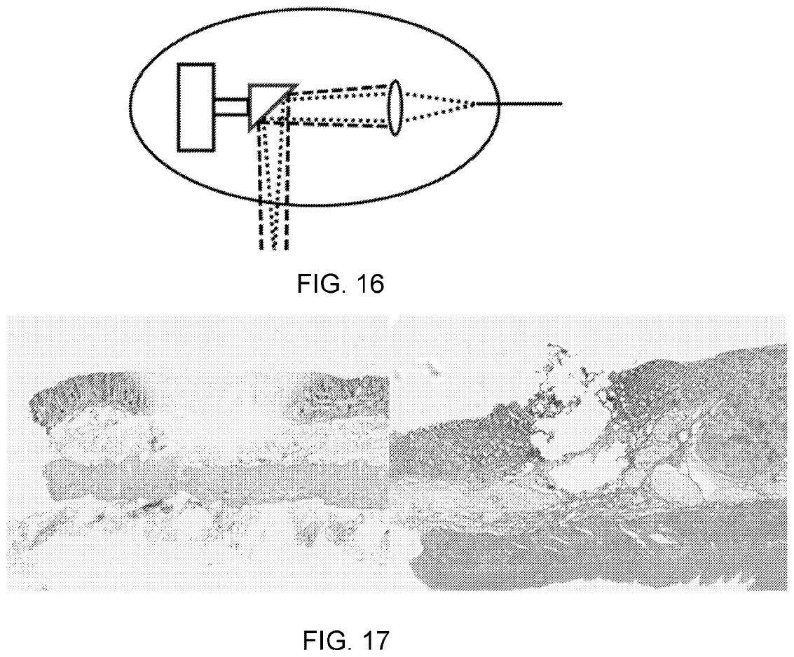

Yet another embodiment of the therapy capsule includes a single waveguide (for both OCT and therapy light), a tunable lens, and a rotational reflector to deliver the light (FIG. 16), where the focusing power of the tunable lens can be controlled. When the capsule is performing OCT imaging, the tunable lens focuses the OCT laser (dotted lines) on the tissue, which may occur when the targeted lesion is identified and before the therapy laser is activated. When delivery of therapeutic light is desired, the tunable lens focus/collimates the therapy light (dashed lines) onto the tissue. The spot size of the therapy laser can also be adjusted by the tunable lens to ablate different size areas on the tissue depending on the characteristics of the lesion.

In various embodiments, the R-TCE OCT-Therapy capsule may be used to screen colon polyps in OCT mode. Once one or more polyps have been identified and located, the system will record the coordinate of the polyp and the rotational reflector will stop and point at the coordinate. The therapy laser may then be activated for a certain period of time with certain illumination patterns, including, e.g., continuous illumination, repeated pulse illumination, etc., to ablate the tissue region.

In certain embodiments, the laser induced tissue ablation can be “non-thermal confined,” which means that the tissue ablation starts from where the therapy laser is focused (e.g., an impingement region) and the size of the area of ablated tissue depends on the thermal transition in the tissue, which can be controlled by the duration of the laser exposure. That is, a portion of the sample beyond the impingement region is ablated and ablation is not limited to the area where laser radiation contacts the tissue. In other embodiments, the laser induced tissue ablation may be “thermal confined,” which means that the tissue ablation happens instantaneously once the laser is turned on and the heat does not transfer outside of the illuminated area, such that the ablation size is determined by the laser spot size on the tissue.

In various embodiments, the wavelength of the therapy laser can be the within the high water-absorption spectrum, which is between 1400 nm-2000 nm. The therapy laser light may be absorbed by the tissue and generate heat to denature the proteins inside the cells or thermally eliminate the cells.

FIG. 17 shows histology images of laser ablated colon tissue (swine colon). The left image was stained with nitroblue tetrazolium chloride (NBTC) to show the inactive cells. The right image was stained with H&E that shows the tissue area eliminated by laser.

### Computer Aided Polyp Detection

In various embodiments, the R-TCE system may be used for obtaining data which can then be used for computer aided polyp detection based on, among other data processing procedures, correlation of the derivative bandwidth, scattering coefficients, angular scattering, and texture analysis.

In some embodiments, three independent data processing approaches can be combined in a data fusion step to generate a classifier. These approaches include: determine the correlation of the derivative (COD) bandwidth, determine the scattering coefficient of each A-line, and determine the angular scattering of the sample. Finally, a classifier (such as ResNet101) may be trained to predict polyp type based on the processed data. In various embodiments, one or more other classifiers can be used including: squeezenet, googlenet, inceptionv3, mobilenetv2, xception, resnet18, resnet50, resnet101, inceptionresnetv2, efficientnetb0, and/or alexnet. In other embodiments, 3D point clouds may be used to plot u_s, ASI, and the standard deviation (STD) of the COD bandwidth, after which boundaries around point clouds which correspond to classes can be identified. The class of an unknown sample could be identified using its position in u_s, ASI, and STD of the COD bandwidths and locations of the boundaries.

### Correlation of the Derivative Bandwidth

An OCT signal contains information about the spectrum of light, and spectroscopic processing methods allow for this depth dependent spectrum to be resolved. Additional processing techniques, such as the COD bandwidth, can be used to assign a scatter size to each tissue pixel in a depth by taking the spectrum at each point, doing a normalize operation (e.g., taking the numerical derivative), then taking the autocorrelation and comparing it to a known reference size. The results can then be represented in 2D or 3D.

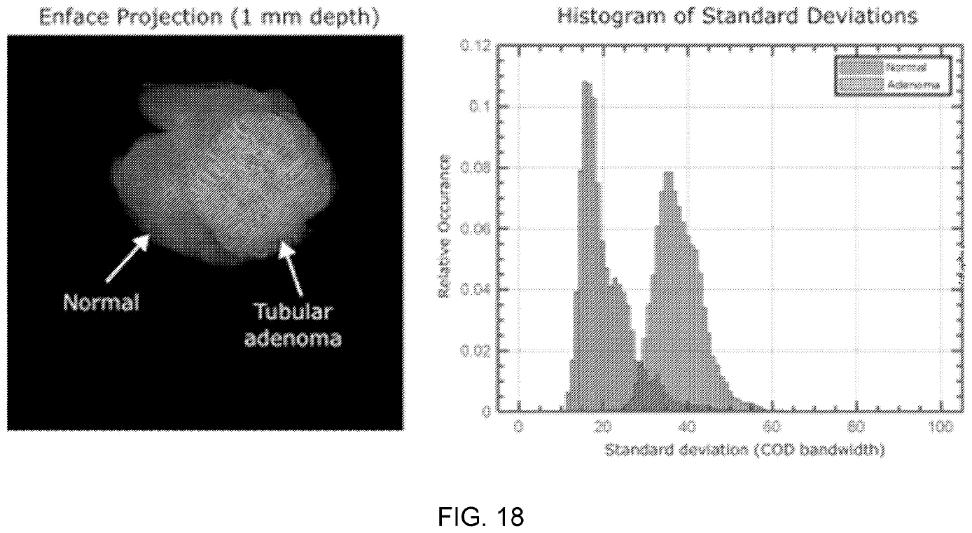

Relative to conventional COD analysis, in the present procedures similar variants are used in which a local standard deviation of the COD bandwidth is computed using a reference square (of arbitrary size) which scans across either a B-scan image or enface map of mean COD bandwidths. At each position during the scan, a standard deviation is computed. This standard deviation gives information about how quickly the modulation frequency varies. The results show that clear differences in normal and adenomatous tissues exist (FIG. 18).

### Scattering Coefficient (μₛ)

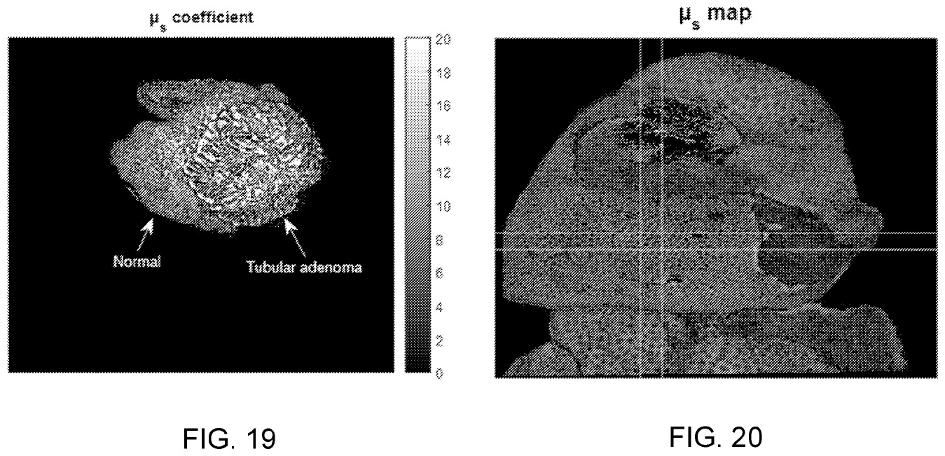

An OCT signal both scatters and attenuates as a function of depth into the sample and this can be described by the scattering and absorption coefficients (μₛ, μₐ respectively), which can be presented on an enface map as a single value for each A-line (FIG. 19, showing an enface map of scattering coefficients). To obtain the scattering and absorption coefficients, the linear OCT image must first be corrected to compensate for intensity decay caused by the confocal gate. Then the us coefficient can be extracted through curve fitting of the A-line or other methods. This is especially useful for obtaining high contrast us maps having the same projection as enface projections, although, they can have higher contrast than enface projections which are generated by simply summing A-lines. Scattering coefficients generated in this manner have been previously shown to be correlated with normal and malignant colorectal tissue.

### Angular Scattering Coefficients

Pit patterns on tissue surfaces have a distinct structure which changes depending on tissue type, which has previously been shown to be useful for discriminating between normal tissue and cancer. Consistent with this, a Fourier domain representation of tissue types indicates differences in frequency components including but not limited to normal tissue, tubular adenomas, hyperplastic polyps, sessile serrated adenomas, and sessile serrated polyps. To extract this information from the Fourier domain spectrum, in one embodiment a scanning square (indicated by intersections of lines in FIG. 20) may be used to extract a subregion of the image. Subsequently, the magnitude spectrum of the Fourier transform (or the logarithm of the Fourier transform) of the subregion may be obtained and then the ratio of the energy in the low/high spatial frequencies in the subregion may be determined so as to produce an index value which in turn can be used to differentiate polyps.

In various embodiments, high contrast enface representations of the data may be used to extract the spatial frequencies. In some embodiments a high contrast enface representation may be obtained by generating enface projections (e.g., which may be obtained by simply summing a well-defined number of pixels from a surface). However, these projections may be low contrast and may not always be oriented with their viewing direction being normal to the tissue surface. Thus, in some embodiments a better option is to generate us maps and then take a patch-wise approach using these maps in which a scanning square is translated across the Us map and the surface orientation for each subregion within the scanning square is corrected. The resulting orientation-corrected patches can then be processed using Fourier domain operations.

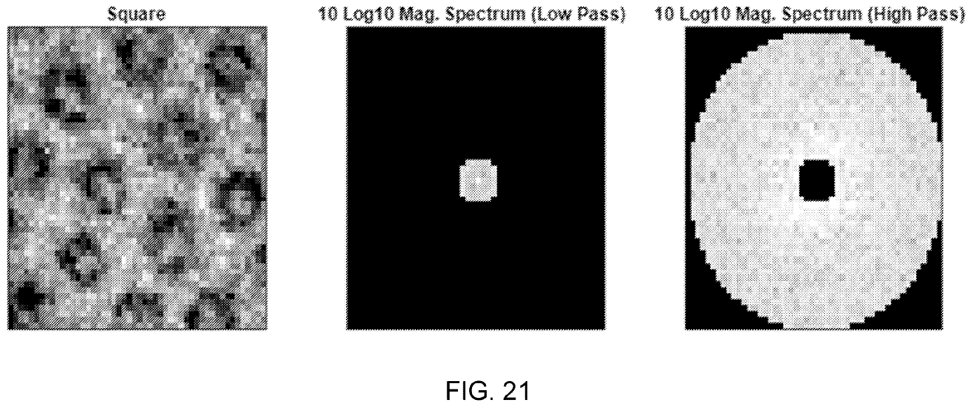

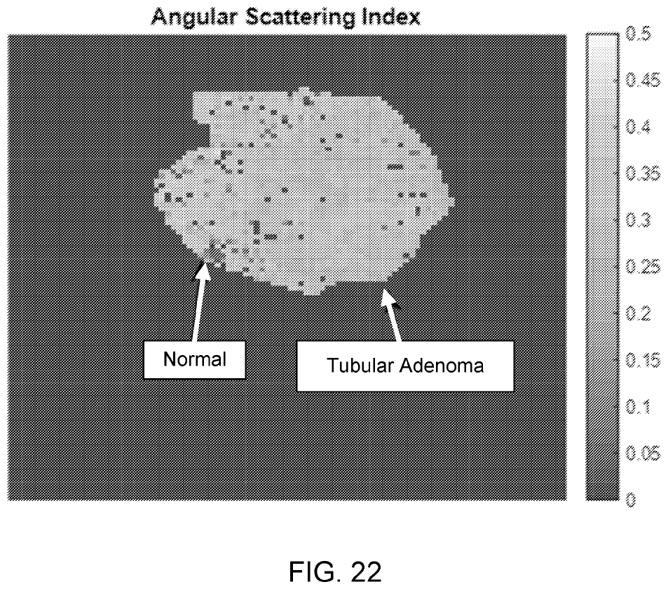

In certain embodiments, a dual aperture approach to this may be taken, where a small circle (low-frequency mask) in Fourier space may be used to pass low spatial frequency components while masking high spatial frequency components (FIG. 21, center panel), and a ring (i.e., a high-frequency mask made of the radial band shown in FIG. 21, right panel) may be used to allow only high spatial frequencies to pass, where a ratio may then be determined of the energies of the low spatial frequency components divided by the energies of the high spatial frequency components. This ratio of energy concentrated in the low/high spatial frequencies gives an angular scattering index (ASI) as shown in FIG. 22. Calculating this index is similar to procedures that have been used for detecting cancer (but not polyps), but with some substantial differences. For one thing, the previous approaches used dynamically assigned ellipsoidal apertures in Fourier space to account for the fact that parts of the image may not be viewed normal to the surface, which likely accounted for how spatial frequencies were represented for imaging tissue tilted relative to the OCT beam. On the other hand, the present procedures instead use a static aperture and a ring in Fourier space to identify the energies to use for determining the index. In the present procedures, each patch can be flattened by first determining its surface normal vector to control tissue orientation.

In various embodiments, the angular or radial energy distribution of either the inner or outer aperture may be used to detect the degree of spatial frequency homogeneity. In still other embodiments, three apertures may be used instead of the two-aperture approach described above, where the three apertures would measure the energy in a low-frequency central portion (as above) as well as in an inner ring and an outer ring (e.g., the radial band in the right panel of FIG. 21 could be divided into two portions, namely an inner ring and an outer ring). The ratio of the energies in the outer ring relative to those in the inner ring may be used as a separate parameter which indicates the concentration of energy in a third band. This additional information is significant insofar as the morphology of hyperplastic and normal tissues may be superficially similar when viewed in real space but the spatial frequencies may be different and these differences in spatial frequency may be discernable in Fourier space.

In other embodiments, similar information can be extracted from B-scans by first attenuation-correcting the OCT signal using the method reported by Cheng (J Biomed Opt, 2019. 24(9): p. 1-17, incorporated herein by reference in its entirety) and then taking a 2D Fourier transform and looking for energy distribution in bands sliced vertically.

### Data Fusion and Machine Learning

In some embodiments, a deep learning network along with patches or full images as training and validation data may be used in two ways: early fusion and late fusion. In early fusion, three channels (Scattering Coefficient maps, Angular Scattering Coefficients, COD bandwidth) are input into a pre-trained network (such as ResNet101) with either polyps or patches left out for validation. In late-stage fusion, each channel is individually fed into a network and a final classification is decided using either weighted probability or a decision tree. Instead of, or in addition to, the channels mentioned above, first and second order intensity statistics including gray level co-occurrence matrices as well as features can also be used.

Upon predicting a polyp type, the system may transmit information about the polyp type to a clinician or other party, who in turn may identify a treatment or other course of action to be taken based on the predicted polyp type.

### Computer Systems

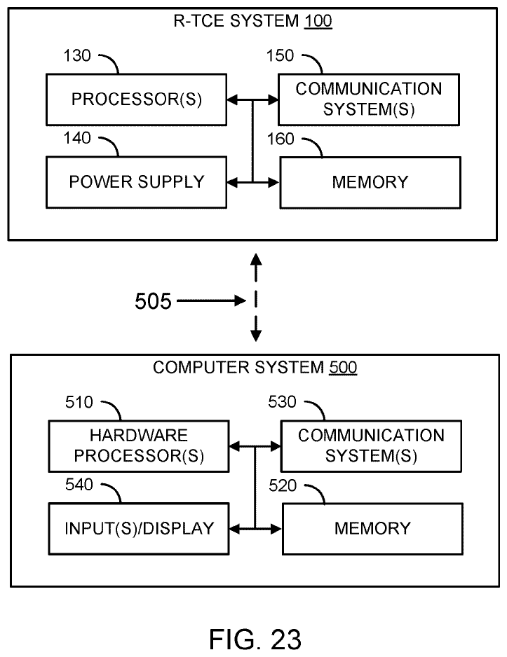

In various embodiments, the R-TCE system 100 (which may include one or more of the drive system, imaging system/console, and/or therapy system/console) may include a controller such as a processor 130, memory 160, a power supply 140 (e.g., a battery such as a rechargeable battery), and/or a communication system 150 (e.g., a wireless communication modality such as Bluetooth or a suitable wired communication mechanism) (FIG. 23). In some embodiments the R-TCE system 100 may be self-contained and may be programmed to carry out various procedures disclosed herein without requiring input or control from an external source. In other embodiments the R-TCE system 100 may be programmed to carry out various procedures disclosed herein which are initiated upon receipt of a signal from an external source. In still other embodiments the R-TCE system 100 may not have the capability to carry out various procedures and instead the procedures may be carried out based on a continuous series of individual commands that are sent to the R-TCE system 100 from an external source.

Thus, in various embodiments the software for controlling the R-TCE system may be present on the R-TCE system 100 itself, may be present on an external device, and/or may be distributed between the R-TCE system 100 and one or more external devices. In certain embodiments the external device may include a standalone computer system 500, which may include a portable device such as a tablet, laptop computer, or a smartphone in communication 505 (e.g., wired or wireless) with the R-TCE system 100 (FIG. 8). The software may be provided as an application (“app”) to run on the device where the app provides instructions to the R-TCE system 100 (e.g., issued via Bluetooth or other wireless or wired communications) to carry out various procedures disclosed herein.

The lower portion of FIG. 23 shows an embodiment of a computer system 500 that can be used to send control information to the R-TCE system 100 (e.g., via wired or wireless communication 505) in accordance with embodiments of the disclosed subject matter. As shown in FIG. 23, in some embodiments, computer system 500 can include a processor 510, a user interface and/or display 540, one or more communication systems 530, and memory 520. In some embodiments, processor 510 can be any suitable hardware processor or combination of processors, such as a central processing unit (CPU), a graphics processing unit (GPU), a microcontroller (MCU), a field programmable gate array (FPGA), an application-specific integrated circuit (ASIC), a dedicated image processor, etc. In some embodiments, input(s) and/or display 540 can include any suitable display device(s), such as a computer monitor, a touchscreen, a television, etc., and/or input devices and/or sensors that can be used to receive user input, such as a keyboard, one or more physical buttons with dedicated functions, one or more physical buttons with software programmable functions, a mouse, a touchscreen, a microphone, a gaze tracking system, motion sensors, etc.

In some embodiments, communications systems 530 can include any suitable hardware, firmware, and/or software for communicating information over a communication network and/or any other suitable communication networks. For example, communications systems 530 can include one or more transceivers, one or more communication chips and/or chip sets, etc. In a more particular example, communications systems 530 can include hardware, firmware and/or software that can be used to establish a Wi-Fi connection, a Bluetooth connection, a cellular connection, an Ethernet connection, an optical connection, etc.

In some embodiments, memory 520 can include any suitable storage device or devices that can be used to store instructions, values, etc., that can be used, for example, by hardware processor 510 to process image data generated by one or more optical detectors, to present content using input(s)/display 540, to communicate with an external computing device via communications system(s) 530, etc. Memory 520 can include any suitable volatile memory, non-volatile memory, storage, any other suitable type of storage medium, or any suitable combination thereof. For example, memory 520 can include RAM, ROM, EEPROM, one or more flash drives, one or more hard disks, one or more solid state drives, one or more optical drives, etc. In some embodiments, memory 520 can have encoded thereon a computer program for carrying out one or more embodiments of the disclosed procedures.

Various embodiments may be carried out with a system that includes a memory (such as memory 520) in communication with a processor (such as processor 510), the memory having stored thereon a set of instructions which, when executed by the processor, cause the processor to carry out steps of various embodiments of the procedures disclosed herein. In some embodiments, the memory may include any suitable computer readable media which can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

### Examples

The following provide non-limiting examples according to various embodiments of the disclosure.

### R-TCE Device Construction and Operation

### Electrical Power to Distal Micro-Motor

Electrically, the micro-motor of the R-TCE device is powered by a motor power unit (MPU) which is integrated within the CIS. The motor power sub-assembly inside the CIS device sends power through the wires. The MPU sub-assembly is powered by a DC-power supply contained within the CIS case. The waveform sent to the capsule catheter micro-motor through the insulated wires has a voltage amplitude of no more than 10.0 V. The bandwidth of the waveform is limited to approximately 10 kHz. The capsule catheter leakage is in compliance with the IEC 60601-1 Standard.

### Electrical Insulation

The R-TCE device includes miniature wires that run through the lumen of the inner sheath and supply power from the motor power unit to the micro-motor. There are three layers of material between the wires and the outer surface of the inner sheath. The wires themselves are insulated with non-conductive perfluoroalkoxy alkane (PFA) material. Surrounding the PFA-enclosed wires is the inner sheath which is composed of multiple layers for insulation. The inner sheath is made of an inner PTFE coating layer and an outer PMMA layer. The inner sheath is enclosed inside of the driveshaft, and an outer sheath which comes into contact with the subject is put over the driveshaft. The outer sheath is made of the same materials as the inner sheath.

The non-conductive capsule body serves as the main layer of insulation for the micro-motor and the wires in the distal tip of the catheter. Solder junctions between the wires and the micro-motor within the capsule body are covered by non-conductive epoxy, providing a second layer of insulation as well.

### Capsule Design

In one embodiment, the basic design of the capsule includes: a rigid capsule body that contains micro-optics, a micro-motor to spin the micro-optics, and a tether that connects to the imaging console. The variation disclosed herein is the Retrograde TCE capsule (FIGS. 1, 1A). R-TCE features a soft/resilient external silicone layer with threads which provides the additional functionality of self-propulsion to navigate the colon while minimizing local tissue damage. The capsule measures 27 mm long including the external layer. The inner rigid capsule has a diameter of 11 mm while the external flexible silicone layer has a diameter of 16 mm at the ends of the threads (FIG. 1A). The external layer is 1 mm thick where there are no threads. Finally, the outer sheath has a diameter of 2.1971 mm.

Design control procedures have been followed for all capsules. The motor power unit design includes failure mode analysis as well as verification and validation procedures to ensure that the device is electrically and mechanically safe on manufacturing and usage levels. A Failure Modes and Effects Analysis has been performed on the capsule resulting in low electrical risk to the subject or the user by following the guidance for medical electrical equipment in NFPA-99 “Standard for Health Care Facilities” and the IEC 60601-1 Standard.

### Specifications

Table 1 lists some key specifications of the R-TCE imaging probe:

TABLE 1
Specifications for R-TCE

| | R-TCE imaging probe | |
|---|---|---|
| Parameter | Typical Value | Unit |
| Light transmission | SMF core illumination | — |
| | SMF core and cladding | |
| | collection | |
| Light transmission range | 1240 to 1380, centered at | nm |
| | 1310 +/− 70 | |
| Transversal Resolution at | 40 +/− 5 | μm |
| 1310 nm | | |
| Working distance from the | 6 | mm |
| capsule outer wall | | |
| Scanning orientation | Side-viewing | — |
| Scanning speed | 20-50 | rps |
| Max. Tether Length | <3 | m |
| Max. Outer Capsule | <13 | mm |
| Diameter | | |
| Max. Capsule Length | 30 | mm |
| Current | AC (rms) <150 | mA |
| | DC (time averaged) <10 | mA |
| DC voltage transport | <10 | V |

### Safety Testing

The following is a table of tests that are conducted on the catheter to ensure the previously mentioned safety features are in place before release for clinical use.

TABLE 2
Safety Testing Parameters

| Feature | Test Name | Pass Criteria |
|---|---|---|
| Low voltage & current | Electrical stability | Confirmation of correct and stable voltage & |
| | | current when the catheter is connected to the micro- |
| | | motor power unit |
| Tether insulation | Tether mechanical | No material deformation upon bending |
| | stability | No defects at interfaces or stretching of tether upon |
| | | straight and angle pull force tests |
| | | No defects in material after mimicking clinical |
| | | use and performing disinfection procedure |
| Capsule insulation | Capsule mechanical | Capsule cap interfaces remain intact upon strength testing |
| | stability | Probe housing interfaces remain intact upon strength |
| | | testing |
| | | No defects in materials or interfaces after mimicking |
| | | clinical use and performing disinfection procedure |
| Negligible electrical | Lead leakage test | Electrical leakage from the catheter is <10 μA using an |
| leakage from catheter | | electrical safety analyzer with lead leakage testing |
| to patient | | capabilities. Test & analyzer are compliant with |
| | | ANSI/AAMI ES1: 1993 (NFPA-99). |

A catheter will be released for clinical use only if all the above tests pass the given criteria. Each capsule is labeled with unique identifier, HLDI date, and expiration date.

### Environmental Requirements

The catheter is designed to withstand normal transportation and normal operating conditions, nevertheless the catheter should be handled with care during transportation and setting up. Banging or dropping the catheter can damage the capsule and could result in the catheter being unavailable for clinical use. The catheter is designed to be operated at ambient temperatures (10-35° C.) and 10-90% relative humidity.

### Pre-Procedure Testing and Calibration

As disclosed herein, each capsule is thoroughly disinfected before its first use as well as after each use. After disinfection, each catheter will be tested and calibrated before use. After passing visual and tactile inspection of integrity, the capsule will be connected to the system for image quality check. If a catheter fails any of those tests, it cannot be used for clinical imaging.

### Optical and Electrical Connection Procedure

1. Connect the Drive System (R-TCE-DS) to the CIS and plug in the CIS to power.
2. Ensure that the Drive System Catheter Cap interface is homed, which should automatically occur when R-TCE-DS is powered (via the CIS).
3. Align the capsule's catheter cap to the drive system catheter cap. The button protruding out of the capsule's outer catheter cap corresponds to the top position of the catheter cap and should be aligned with the drive system as such.
4. Gently push the catheter cap into the drive system until you hear a click to ensure locking. The Catheter Cap should not be able to rotate or be easily pulled out if correctly inserted.

### Process for Handling During Clinical Procedures

1. Straighten out the driveshaft to hand it to the capsule operator-make sure that the driveshaft has no knots and is not twisted.
2. The capsule operator can hold the catheter in their fingers at any location of the driveshaft or the capsule making sure not to apply too much pressure.
3. The catheter can be handed to the subject who can hold the driveshaft in their fingers at any location of the tether or the capsule making sure not to apply too much pressure.
4. The capsule operator can control the capsule position throughout the procedure via the driveshaft. The driveshaft can withstand bending but should not be torqued significantly due to the fragile optical fiber inside.
5. Capsule position can be estimated using location marks on the outer sheath.
   Navigation with Capsule
1. During the procedure, the live imaging should be monitored on the CIS. If the image begins to “tumble” (stops rotating, then quickly rotates for a few seconds) or stops completely, the operator should start counting or watch a clock. This is indication of the capsule failing to rotate or progress/advance.
2. If the problem persists for 15 seconds, the rotation will be stopped and appropriate steps will be taken. This may include manually rotating the driveshaft to relieve the torque or putting the drive system in reverse and pulling the capsule out completely.

### Optical and Electrical Disconnection

1. Disable laser on the Imaging System and Turn off the Micro-motor power unit.
2. Gently release the locking mechanism and pull the capsule's catheter cap away from the drive system.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A system for performing retrograde tethered capsule endomicroscopy, comprising:
a capsule comprising at least one outwardly-facing helical thread;
a drive shaft coupled to the capsule and configured to rotate the capsule; and
an optical system disposed within the capsule and configured to obtain circumferential imaging information.

2. The system of claim 1, further comprising an optical waveguide disposed within the drive shaft, and
wherein the optical system comprises a lens optically coupled to the optical waveguide and disposed within the capsule.

3. The system of claim 2, wherein the optical system further comprises an angled reflector aligned with the lens, and
wherein the angled reflector is configured to rotate to direct a beam of electromagnetic radiation emitted from the lens in a circumferential pattern around the capsule.

4. The system of claim 3, wherein the capsule further comprises a circumferential imaging window aligned with the angled reflector.

5. The system of claim 4, wherein the at least one outwardly-facing helical thread is absent in a region of the circumferential imaging window.

6. The system of claim 5, wherein the capsule comprises a rigid inner capsule body and a resilient outer portion, and
wherein the resilient outer portion comprises the at least one outwardly-facing helical thread.

7. The system of claim 6, wherein the resilient outer portion comprises a proximal layer and a distal layer attached to the rigid inner capsule body,
wherein the circumferential imaging window is between the proximal layer and the distal layer.

8. The system of claim 6, wherein the resilient outer portion comprises silicone.

9. The system of claim 3, further comprising an imaging system,
wherein the imaging system is coupled to the optical system using the optical waveguide.

10. The system of claim 9, wherein the imaging system comprises an interferometric imaging system, and
wherein the circumferential imaging information is obtained using the interferometric imaging system.

11. The system of claim 1, further comprising a drive system coupled to the drive shaft.

12. The system of claim 11, wherein the drive shaft comprises a semirigid drive shaft, and
wherein the drive system is configured to rotate the semirigid drive shaft to rotate the capsule in a first rotational direction and a second rotational direction opposite the first rotational direction.

13. The system of claim 12, wherein the drive system is coupled to the semirigid drive shaft using a catheter cap,
wherein the catheter cap provides at least one of optical or electrical connections to the capsule.

14. The system of claim 13, wherein the catheter cap comprises an inner cap disposed within an outer cap with a bearing therebetween,
wherein the inner cap rotates within the outer cap via the bearing to rotate the semirigid drive shaft.

15. The system of claim 3, wherein the angled reflector is rotated using a micro-motor,
wherein power is provided to the micro-motor via wires in the drive shaft.

16. The system of claim 15, wherein the lens comprises at least one of a ball lens or a prism configured to direct light from the optical waveguide toward the angled reflector.

17. The system of claim 9, further comprising a balloon surrounding the drive shaft adjacent to the capsule.

18. The system of claim 17, wherein the balloon is configured to be inflated during operation of the imaging system.

19. The system of claim 4, further comprising an infusion tube configured to deliver fluid to the imaging window to remove debris.

20. The system of claim 1, further comprising a first electromagnetic radiation source for imaging and a second electromagnetic radiation source for ablation,
wherein each of the first electromagnetic radiation source and the second electromagnetic radiation source are optically coupled to the optical system,
wherein the first electromagnetic radiation source is configured to obtain the circumferential imaging information of a sample, and
wherein the second electromagnetic radiation source is configured to ablate the sample.

21. The system of claim 20, wherein the optical system comprises a lens configured to at least one of focus the electromagnetic radiation from the first electromagnetic radiation source or collimate the electromagnetic radiation from the second electromagnetic radiation source.

22. The system of claim 21, further comprising an optical waveguide disposed within the drive shaft, and
wherein each of the first electromagnetic radiation source and the second electromagnetic radiation source deliver electromagnetic radiation to the optical system using the optical waveguide.

23. The system of claim 22, wherein the lens comprises a tunable lens, and
wherein the tunable lens is configured to change a focusing power based on whether the first electromagnetic radiation source or the second electromagnetic radiation source is delivering electromagnetic radiation to the optical system.

24. The system of claim 21, further comprising a first optical waveguide and a second optical waveguide disposed within the drive shaft, and
wherein the first electromagnetic radiation source delivers electromagnetic radiation to the optical system using the first optical waveguide, and
wherein the second electromagnetic radiation source delivers electromagnetic radiation to the optical system using the second optical waveguide.

25. The system of claim 1, wherein the capsule is pretreated with plasma to reduce fouling during use.

26. The system of claim 25, wherein the capsule comprises polymethyl methacrylate (PMMA) and is pretreated with a O₂/CF₄ plasma to etch a wall of the capsule.

27. A method for performing retrograde tethered capsule endomicroscopy, comprising:
providing a capsule comprising at least one outwardly-facing helical thread and an optical system disposed within the capsule;
rotating the capsule using a drive shaft coupled to the capsule; and
obtaining circumferential imaging information using the optical system disposed within the capsule.

28. The method of claim 27, wherein providing a capsule further comprises:
providing the capsule comprising an optical waveguide disposed within the drive shaft,
wherein the optical system comprises a lens optically coupled to the optical waveguide and disposed within the capsule.

29. The method of claim 28, wherein providing a capsule further comprises:
providing the capsule wherein the optical system further comprises an angled reflector aligned with the lens, and
wherein the method further comprises:
rotating the angled reflector to direct a beam of electromagnetic radiation emitted from the lens in a circumferential pattern around the capsule.

30. The method of claim 29, wherein providing a capsule further comprises:
providing the capsule wherein the capsule further comprises a circumferential imaging window aligned with the angled reflector, and
wherein the at least one outwardly-facing helical thread is absent in a region of the circumferential imaging window.

31. The method of claim 30, wherein providing a capsule further comprises:
providing the capsule wherein the capsule further comprises a rigid inner capsule body and a resilient outer portion,
wherein the resilient outer portion comprises the at least one outwardly-facing helical thread.

32. The method of claim 31, wherein providing a capsule further comprises:
providing the capsule wherein the resilient outer portion comprises a proximal layer and a distal layer attached to the rigid inner capsule body,
wherein the circumferential imaging window is between the proximal layer and the distal layer.

33. The method of claim 29, wherein obtaining circumferential imaging information further comprises:
obtaining circumferential imaging information using an imaging system,
wherein the imaging system comprises an interferometric imaging system, and
wherein the imaging system is coupled to the optical system using the optical waveguide.

34. The method of claim 27, wherein providing a capsule further comprises:
providing the capsule further comprising a drive system coupled to the drive shaft,
wherein the drive shaft comprises a semirigid drive shaft, and
wherein the method further comprises:
rotating the semirigid drive shaft using the drive system to rotate the capsule in a first rotational direction and a second rotational direction opposite the first rotational direction.

35. The method of claim 34, wherein providing a capsule further comprises:
providing the capsule wherein the drive system is coupled to the semirigid drive shaft using a catheter cap,
wherein the catheter cap provides at least one of optical or electrical connections to the capsule.

36. The method of claim 35, wherein providing a capsule further comprises:
providing the capsule wherein the catheter cap comprises an inner cap disposed within an outer cap with a bearing therebetween, and
wherein the method further comprises:
rotating the inner cap within the outer cap via the bearing to rotate the semirigid drive shaft.

37. The method of claim 29, wherein rotating the angled reflector further comprises:
rotating the angled reflector using a micro-motor,
wherein power is provided to the micro-motor via wires in the drive shaft.

38. The method of claim 37, wherein obtaining circumferential imaging information further comprises:
directing light from the optical waveguide toward the angled reflector using the lens,
wherein the lens comprises at least one of a ball lens or a prism.

39. The method of claim 33, wherein providing a capsule further comprises:
providing the capsule further comprising a balloon surrounding the drive shaft adjacent to the capsule, and
wherein the method further comprises:
inflating the balloon during operation of the imaging system.

40. The method of claim 30, wherein providing a capsule further comprises:
providing the capsule comprising an infusion tube, and
wherein the method further comprises:
delivering fluid to the imaging window using the infusion tube to remove debris.

41. The method of claim 27, wherein providing a capsule further comprises:
providing the capsule comprising a first electromagnetic radiation source for imaging and a second electromagnetic radiation source for ablation,
wherein each of the first electromagnetic radiation source and the second electromagnetic radiation source are optically coupled to the optical system, and wherein the method further comprises:
obtaining the circumferential imaging information of a sample using the first electromagnetic radiation source, and
ablating the sample using the second electromagnetic radiation source.

42. The method of claim 41, wherein providing a capsule further comprises:
providing the capsule wherein the optical system comprises a lens, and
wherein the method further comprises at least one of:
focusing the electromagnetic radiation from the first electromagnetic radiation source using the lens, or
collimating the electromagnetic radiation from the second electromagnetic radiation source using the lens.

43. The method of claim 42, wherein providing a capsule further comprises:
providing the capsule with an optical waveguide disposed within the drive shaft, and
wherein the method further comprises:
delivering electromagnetic radiation from each of the first electromagnetic radiation source and the second electromagnetic radiation to the optical system using the optical waveguide.

44. The method of claim 43, wherein providing a capsule further comprises:
providing the capsule wherein the lens comprises a tunable lens, and
wherein the method further comprises:
changing a focusing power of the tunable lens based on whether the first electromagnetic radiation source or the second electromagnetic radiation source is delivering electromagnetic radiation to the optical system.

45. The method of claim 42, wherein providing a capsule further comprises:
providing the capsule further comprising a first optical waveguide and a second optical waveguide disposed within the drive shaft, and
wherein the method further comprises:
delivering electromagnetic radiation from the first electromagnetic radiation source to the optical system using the first optical waveguide, and
delivering electromagnetic radiation from the second electromagnetic radiation source to the optical system using the second optical waveguide.

46. The method of claim 27, further comprising:
pretreating the capsule with plasma to reduce fouling during use,
wherein the capsule comprises polymethyl methacrylate (PMMA) and is pretreated with a O₂/CF₄ plasma to etch a wall of the capsule.

47. The method of claim 41, wherein ablating the sample further comprises:
directing electromagnetic radiation from the second electromagnetic radiation source at an impingement region of the sample for a particular duration of time to perform non-thermal confined ablation,
wherein a portion of the sample beyond the impingement region is ablated.

48. The method of claim 41, wherein ablating the sample further comprises:
directing electromagnetic radiation from the second electromagnetic radiation source at an impingement region of the sample to perform thermal confined ablation,
wherein no portion of the sample beyond the impingement region is ablated.

49. A method for polyp detection, comprising:
obtaining, using a processor, an image of a sample;
determining, using the processor, a correlation of the derivative (COD) bandwidth for the image;
determining, using the processor, a scattering coefficient for the image;
determining, using the processor, an angular scattering coefficient for the image; and
training, using the processor, a classifier based on the COD bandwidth, the scattering coefficient, and the angular scattering coefficient to predict a polyp type in the sample.

50. The method of claim 49, wherein determining a COD bandwidth further comprises:
determining the COD bandwidth for each of a plurality of reference squares of the image comprising:
determining a local standard deviation for the COD bandwidth for each of the plurality of reference squares of the image.

51. The method of claim 49, wherein the image comprises a plurality of A-lines; and
wherein determining a scattering coefficient further comprises:
determining a scattering coefficient for each of the plurality of A-lines of the image.

52. The method of claim 51, wherein determining a scattering coefficient for each of the plurality of A-lines of the image further comprises:
determining the scattering coefficient for each of the plurality of A-lines of the image using curve fitting.

53. The method of claim 49, wherein determining an angular scattering coefficient further comprises:
generating a scattering coefficient map based on the image,
generating a plurality of subregions of the scattering coefficient map,
adjusting an orientation of each of the plurality of subregions of the scattering coefficient map to be normal to a surface of the sample,
determining energies associated with low spatial frequency regions and high spatial frequency regions of each of the plurality of subregions of the scattering coefficient map, and
generating a ratio of the energies associated with the low spatial frequency regions and the high spatial frequency regions of each of the plurality of subregions of the scattering coefficient map to generate an angular scattering index for each of the plurality of subregions of the scattering coefficient map.

54. The method of claim 53, wherein determining energies associated with low spatial frequency regions and high spatial frequency regions of each of the plurality of subregions of the scattering coefficient map further comprises:
generating a Fourier domain magnitude spectrum for each of the plurality of subregions of the scattering coefficient map,
determining the energies associated with the low spatial frequency regions by applying a low-frequency mask to the Fourier domain magnitude spectrum for each of the plurality of subregions of the scattering coefficient map, and
determining the energies associated with the high spatial frequency regions by applying a high-frequency mask to the Fourier domain magnitude spectrum for each of the plurality of subregions of the scattering coefficient map.

55. The method of claim 49, wherein the classifier comprises ResNet101.

56. The method of claim 49, wherein obtaining an image of a sample further comprises:
obtaining the image of the sample using an R-TCE system.

57. The method of claim 49, wherein the sample comprises colon tissue.

58. The method of claim 49, further comprising:
identifying a treatment based on predicting the polyp type.

59. The method of claim 49, further comprising:
transmitting information about the polyp type to a clinician.
